(19) United States
(12) Patent Application Publication
(10) Pub. No.: US 2007/0088080 A1
(43) Pub. Date: Apr. 19, 2007

(54) Gsk-3 inhibitors isolated from marine organisms

(75) Inventors: Diana Alonso Gordillo, Madrid (ES); Isabel Dorronsoro Diaz, Madrid (ES); Ana Martinez-Gil, Madrid (ES); Gema Panizo Del Pliego, Madrid (ES); Ana Fuertes Huerta, Madrid (ES); Ma Jose Perez Puerto, Madrid (ES); Ester Martin Aparicio, Madrid (ES); Dario Perez Navarro, Madrid (ES); Miguel Medina Padilla, Madrid (ES)

Correspondence Address: FISH & RICHARDSON PC, P.O. BOX 1022, MINNEAPOLIS, MN, 55440-1022, US

(73) Assignee: NEUROPHARMA, S.A., Madri (ES)

(21) Appl. No.: 10/596,188

(22) PCT Filed: Dec. 2, 2004
(86) PCT No.: PCT/GB04/50033

§ 371 Date: Sep. 14, 2006

(30) Foreign Application Priority Data
Dec. 2, 2003 (GB) 0327908.0

(51) Int. Cl. A61K 31/365; C07D 405/02

(52) U.S. Cl. 514/473; 549/314

(57) ABSTRACT

The present invention provides the use of a compound of formula (I); wherein: n is 0, 1, 2, or 3, the bonds shown with the dashed lines are saturated, or unsaturated with one or more double bonds; R, and R′are independently selected from hydrogen, alkyl, aryl, —OH, —OR″ —SH, —SR″—NH, —NHR″, ═O, ═NH, ═NR″; R″ is independently selected from alkyl, aryl, in the preparation of a medicament for the treatment of a disease requiring a GSK-3 inhibitor. Also provided is a compound of formula (II); wherein n is 0, 1, 2, or 3 the bonds shown with the dashed lines are saturated, or unsaturated with one or more double bonds. Also provided are methods of treating chronic neurodegenerative conditions.

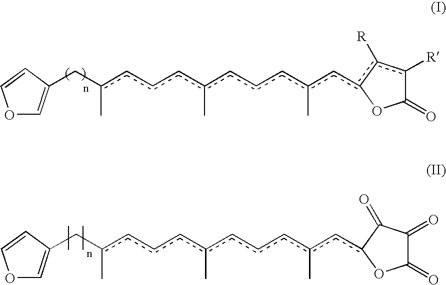

The present invention relates to GSK-3 inhibitors, and in particular to GSK-3 inhibitors isolated from marine organisms. The inhibitors are of use for treating Alzheimer's disease and other conditions.

### BACKGROUND OF THE INVENTION

Alzheimer's disease is characterized by the development of senile plaques and neurofibrillary tangles, which are associated with neuronal destruction, particularly in cholinergic neurons. Neurofibrillary tangles are structures formed by paired helical filaments (PHFs). They are comprised mainly of microtubule-associated protein (MAP) tau in an abnormally hyperphosphorylated state. Such aberrant phosphorylation of tau; determined by the effects of different protein kinases and phosphatases, appears to compromise on its ability to bind to and stabilise microtubules and this may contributes to AD pathology. Thus, the blockade of this hyperphosphorilation step may be a prime target at which to interrupt to pathogenic cascade.

The selective inhibitors of tau kinases might be new effective drugs for the treatment of AD. The search for tau kinases inhibitors is a field of a great interest. Tau can be phosphorylated by several prolines-directed kinases (PDKs) and non-PDKs. However in AD the extra role of any of this kinases in the abnormal hyperphosphorylation of tau is not yet understood and to date, the activity of these kinases has not been found to be up-regulated. There is no doubt that GSK-3β is an in vivo kinase in the brain. These findings open the gate to the use of GSK-3β inhibitors as therapeutic agents in treatment of AD. GSK-3 is involved not only in neurodegenerative diseases as Alzheimer's disease, but also in diabetes type II, cancer, inflammation process and some other unmet disorders.

The oceans are the source of a large group of structurally unique natural products that are mainly accumulated in invertebrates such as sponges, tunicates, bryozoans, and mollusc [Burkhard Haefaer, Drug Dis. Today. Aug. 2, 2003, 536-544]. Several of these compounds show pronounced pharmacological activities and are interesting candidates for new drugs in several areas of treatment [D J. Newman, G M. Cragg, I M. Snader, J. Nat. Prod. 2003, 66, 1022-1037]. Once again sponges have provided marine natural products than any order phylum, due in part to their propensity to produce bioactive metabolites [J. Faulkner, Nat. Prod. Rep. Jan. 19, 2002, 19, 1-48].

At the moment few marine compounds are known as GSK-3 inhibitors: hymenialdisine [Meijer, L. et al. Chem. Biol. 2000, 7, 51-63] has been isolated from marine organisms.

### SUMMARY OF THE INVENTION

In an effort to find selective inhibitors of GSK-3β, we have investigated the marine sponge Ircinia sp. [G. Alfano, G. Cimino and S. De Stefano. Tetrahedron. 1972, 28, 333]. In the course of our research, we found that isopropanolic extracts from sponges of genus Ircinia (species dendroides, variabilis and oros) showed potent inhibition of GSK-3β. Fractionation and purification of active components from these extracts, guided by a GSK-3 inhibition assay, resulted in the isolation of terpenoids as new GSK-3 inhibitors with potential use as therapeutic agents. Synthetic analogues can then be designed.

According to the present invention, we provide organic solvent extracts of the sponge Ircinia sp., and specially Ircinia dendroides, Ircinia variabilis and Ircinia oros, which show activity as potent GSK-3 inhibitors; the furanoterpenoids of formula I either isolated from these extracts or synthetically prepared, which also show activity as potent GSK-3 inhibitors, especially Palinurin (1); and novel furanoterpenoids of formula II, which also show activity as potent GSK-3 inhibitors, especially Tricantin (2). These compounds are useful in the treatment of diseases in which GSK-3 is involved, mainly chronic neurodegenerative conditions including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, taupathies such as Pick's disease, corticobasal degeneration, supranuclear palsy, etc, frontotemporal dementia, Huntington's disease, AIDS associated dementia, amyotrophic lateral sclerosis, multiple sclerosis, diabetes, especially diabetes type II, and conditions associated with diabetes, neurotraumatic diseases such as acute stroke, mood disorders such as schizophrenia and bipolar disorders, promotion of functional recovery post stroke, cerebral bleeding (for example, due to solitary cerebral amyloid angiopathy), hair loss, obesity, atherosclerotic cardiovascular disease, hypertension, polycystic ovary syndrome, syndrome X, ischaemia, traumatic brain injury, cancer, leukopenia, Down's syndrome, Lewy body disease, cancer and hyperproliferative diseases as hyperplasias, metaplasias, displasias and immunodeficiency, psoriasis, arteriosclerosis or restenosis; and inflammation and chronic inflammatory processes,

For example, we provide compounds of formula (I) or (II), pharmaceutical compositions which contain such a compound and a pharmaceutically acceptable carrier, methods of treatment which employ such compounds or compositions, processes for preparing pharmaceutical compositions, and the use of the compounds in the preparation of medicaments for use in the treatment methods. The pharmaceutical compositions, also referred to as medicaments, may be designed for oral administration as solid unit doses using conventional pharmaceutical auxiliary materials.

### DETAILED DESCRIPTION OF THE INVENTION

Some sesterterpenes isolated from the sponge Ircinia sp. display a wide range of bioactivities, including cytotoxicity [Hamad H. Issa, Junichi Tanaka, Tatsuo Higa, J. Nat. Prod. 2003, 66, 251-254], action as protein kinase inhibitor [Malcolm S. Buchanan, Annette Edser, J. Nat. Prod. 2001, 64, 300-303] and antibiotic effect [John Faulkner, Tetrahedron Lett. 1973, 39, 3821-3822]. Palinurin has been described as an anti-inflammatory and antibacteraal compound [M. T. Hamann, J. Org. Chem. 1999, 64, 1258-1260].

The present inventors have discovered, after thorough research, that organic solvent extracts, in particular the isopropanolic extracts, of the Ircinia sp., in particular Ircinia dendroides, Ircinia variabilis and Ircinia oros collected from the Mediterranean Sea, showed potent inhibition of GSK-3β (90% of inhibition at 50 mg/ml).

The isopropanolic extract was partitioned between water and diethyl ether, and the organic layer was where the inventors found the inhibition of GSK-3β.

The purification of this fraction yielded the isolation of different compounds within the general formulae I and II. 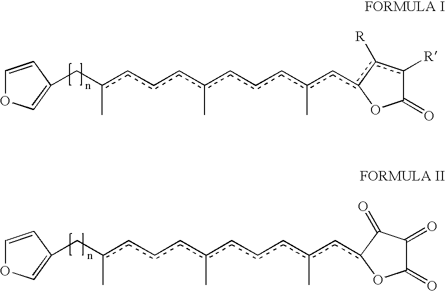
where:
n is 0, 1, 2, or 3
the bonds shown with the dashed lines are saturated, or unsaturated with one or more double bonds;
R, and R′ are independently selected from hydrogen, alkyl, aryl, —OH, —OR′, —SH, —SR″, —NH₂, —NHR″, ═O, ═NH, ═NR″;
R″ is independently selected from alkyl, aryl.

In the above definition of compounds of formula I the following terms have the meaning indicated:

“Alkyl” refers to a straight or branched hydrocarbon chain radical consisting of carbon and hydrogen atoms, containing no saturation, having one to eight carbon atoms, and which is attached to the rest of the molecule by a single bond, e.g., methyl, ethyl, n-propyl, i-propyl, n-butyl, t-butyl, n-pentyl, etc. Alkyl radicals may be optionally substituted by one or more substituents independently selected from the group consisting of halo, hydroxy, alkoxy, carboxy, cyano, carbonyl, acyl, alkoxycarbonyl, amino, nitro, mercapto and alkylthio.

“Aryl” refers to a phenyl naphthyl, indenyl, fenanthryl or anthracyl radical, preferably phenyl or naphthyl radical. The aryl radical may be optionally substituted by one or more substituents selected from the group consisting of hydroxy, mercapto, halo, alkyl, phenyl, alkoxy, haloalkyl, nitro, cyano, dialkylamino, aminoalkyl, acyl and alkoxycarbonyl, as defined herein.

Preferably n is 3.

Preferably the carbon chain shown with the dashed line is saturated with carbon-carbon bonds (sp³C-sp³C) or unsaturated with one or more double bonds which are E-double carbon-carbon bonds (sp²C-sp²C), or Z-double carbon-carbon bonds (sp²C-sp²C). In particular for the carbon chain (excluding the ring) it is preferred to have two or more double bonds , especially two double bonds which are conjugated. Most preferred is to have double bonds giving the partial structure:

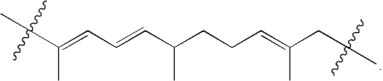

Furthermore, in the substituted tetrahydrofuran ring of formula (I), the preferred structure is:

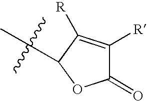

Preferably R is —OH or ═O.

Preferably R′is ═O or methyl.

Alkyl groups preferably have 1 to 6 carbon atoms, more preferably 1 to 3 carbon atoms, such as methyl, ethyl or propyl.

Aryl groups preferably are phenyl or naphthyl.

Chemical elucidation of two of the compounds isolated from Ircinia sp. revealed structures 1 and 2. These furanosesquiterpenoids derivatives showed potent inhibition on GSK-3.

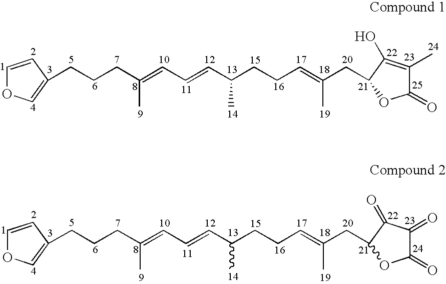

Compound 2 is a new chemical structure not reported until date.

Such compounds, and the other compounds of the general formulae (I) and (II), can be prepared by synthesis.

The present invention is in particular directed to the use of the extracts and compounds obtained from the marine organism Ircinia sp. in the treatment of diseases in which GSK-3 is involved, as well as to the novel compounds of general formula II, in particular compound 2, isolated from the marine organism Ircinia sp.

### EXAMPLES OF THE INVENTION

1. Activity of the Compounds of the Invention:

1.1. Experimental Procedures

1.1.1. GSK3β Inhibition

Recombinant human glycogen synthase kinase 3B was assayed in MOPS 8 mM pH7.3, EDTA 0.2 mM, MgCl. 10 mM and sodium orthovanadate 0.25 mM in the presence of 62.5 μM of Phospho-Glycogen Synthase Peptide-2 (GS-2), 0.5 μCi γ-³³P-ATP and unlabelled ATP at a final concentration of 12.5 μM. The final assay volume was 20 μl. After incubation for 30 minutes at 30° C., 15 μl aliquots were spotted onto P81 phosphocellulose papers. Filters were washed four times for at least 10 minutes each and counted with 1.5 ml of scintillation cocktail in a scintillation counter.

Marine extracts and fractions were routinely tested at a single concentration of 25 μg/ml whereas isolated compounds Palinurin and Tricantin IC50 values were calculated analyzing inhibition curves by non-linear regression using GraphPad Prism.

1.1.2. Inhibition of Tau Phosphorylation Human neuroblastoma SHSY5Y cells were seeded in 96-well plates (25000 cells/well) in Dulbecco's modified Eagle's medium (DMEM) supplemented with 10% fetal calf serum. One day later, cells were treated with samples for 24 h at 37° C. After treatment, cultures were washed with phosphate-buffered saline and lysed for 30 min at 4° C. in 120 μl of extraction buffer (10 mM Tris-HCl, pH 7.4, 100 mM NaCl, 1 mM EDTA, 2 mM Na₃VO₄, 1% Triton X-100, 10% glycerol, 0.1% SDS, 0.5% Sodium deo.ycholate, 1 mM PMSF and a protease inhibitor cocktail.

The quantitative definition of phosphorylated human Tau was made taking 40 μl of the cell lysate and using a phosphorylation-specific antibody against Tau [pS396] in a sandwich ELISA. Tau phosphorylation was estimated by measuring of absorbance at 450 nm in a microtiter plate reader.

In addition to tau phosphorylation assays, quantification of cell death and cell lysis was made by measuring LDH release. For the quantitative determination of cell survival, 40 μl of the cell lysate were incubated with an equal volume of reaction mixture at room temperature for 20-30 mm. The measure of absorbance was made in a microtiter plate reader with 490-492 nm filter.

1.2. Biological Activity

1.2.1. GSK3β Inhibition

Different Ircinia dendroides extracts were tested at concentrations ranging from 5 to 50 μg/ml on the in vitro GSK3β assay. In these assay Ircinia dendroides extracts inhibit recombinant human GSK3 at concentrations over 15 μg/ml.

Kinetic analyses for isolated compounds were performed and the results show that Tricantin inhibits recombinant human GSK3β with an IC50 value of 7.5 μM, whereas Palinurin shows an IC50 value of 4.5 μM.

In order to investigate the mechanism of inhibition of GSK3β by Palanurin, several kinetic experiments were performed varying both ATP and inhibitor concentrations. Preliminary experiments with Palinurin suggest that it might act as a non As-competitive inhibitor.

1.2.2. Inhibition of Tau Phosphorylation

Different Ircinia dendroides extracts were tested at concentrations ranging from 5 to 50 μg/ml on the in vivo phosphorylation assay. In these assays Ircinia dendroides extracts inhibit tau phosphorylation at concentrations over 50 μg/l.

When assayed on the tau phosphorylation cellular assay, both Palinurin and Tricantin showed significant inhibition at a concentration of 200 μM.

2. Physical Properties of the Compounds of the Invention:

The chemical structure elucidation of the compounds were performed in basis to their spectrospical (IR, ES+, WTV, ¹H-NMR and ¹³C-NMR) data together with the results of bidimensional NMR experiments, both homonuclear, such COSY and NOESY pulse sequences, and hetereonuclear experiments such HMQC and HMBC pulse sequences.

The IR, ES+, WV, ¹H-NMR and ¹³C-NMR spectrum of compound 1 showed the same bands of absorptions and signals (chemical shifts and coupling constants) than those was described by G. Alfano in 1979 as Palinurin.

So, here we described Palinurin as a potent GSK-3 inhibitor.

Compound 2 showed the same [M+H]+ ion at m/z 399 and the UVλₘₐₓ at 241 nm that compound 1.

The IRvₘₐₓ spectrum of Compound 2 showed one absorption band at 1724 cm⁻¹ attributable to carbonyl group while the band at 1756 cm⁻¹ attributable to double bond groups that appeared in Compound 1 is not shown in IR spectrum of 2.

The ¹H-NMR spectrum of compound 2 (Table 1) was almost identical to that of compound 1, with the exception that the oxamethynic proton (CH-21) which appeared in the spectrum of 1 at δ 4.8 μm were replaced in 2 by a signal downfield at δ 4.38 μm. The signal corresponding to the CH₃-24 of 1 which appeared at δ 1.64 μm were disappeared in the spectrum of 2. This first evidence led to the suggestion that the change was being in the tetronic acid residue, and it was confirmed when compared the ¹³C-RMN chemical shifts of compounds 1 and 2.

The carbon signal of CH₃-24 was also not observed in compound 2, and the chemical shifts of C-21, C-22 and C-23 was changed respected to Compound 1 (see table 1). The two carbon atoms C22 and C23 appeared at the same chemical shift δ 177.92 μm than C24, this fact allowed us to assign the three carbonyl groups for our novel compound, these evidences led to the suggestion of structure 2. As this new compound was isolated in Tres Cantos, we have named it Tricantin.

The shielded of C-21 is due to the anisotropy environment of carbonyl group in C-22 and the differences in chemical shifts of C-22 and C-23 were due to the presence of two new carbonyl groups. The structure of compound 2 was confirmed using a commercial compound (dehydroascorbic acid) as reference, in which we could observe that the three carbonyl groups appeared at the same chemical shifts as our novel compound. The stereochemistry of Tricantin is not yet assigned.

TABLE 1
Compound 1

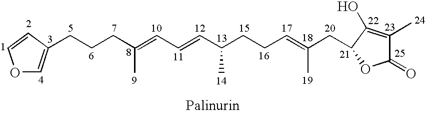

Compound 2

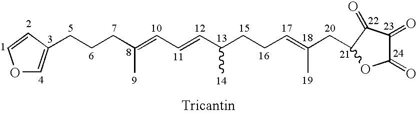

| | Palinurin | | Tricantin | |
|---|---|---|---|---|
| Atom | ¹H-NMR | ¹³C-NMR | ¹H-NMR | ¹³C-NMR |
| 1 | 7.36(s) | 143.90 CH | 7.38(s) | 144.01 CH |
| 2 | 6.28(s) | 111.97 CH | 6.29(s) | 111.98 CH |
| 3 | | 126.31 C | | 126.38 C |
| 4 | 7.23(s) | 140.12 CH | 7.25(s) | 140.18 CH |
| 5 | 2.39(t) | 25.30 CH₂ | 2.38(t) | 25.29 CH₂ |
| 6 | 1.70(m) | 29.52 CH₂ | 1.69(m) | 29.56 CH₂ |
| 7 | 2.06(t) | 40.43 CH₂ | 2.10(t) | 40.43 CH₂ |
| 8 | | 136.80 C | | 136.80 C |
| 9 | 1.71(s) | 17.27 CH₃ | 1.71(s) | 16.57 CH₃ |
| 10 | 5.75(d) | 126.71 CH | 5.77(d) | 126.71 CH |
| 11 | 6.20(dd) | 126.69 CH | 6.21(dd) | 126.69 CH |
| 12 | 5.38(dd) | 139.09 CH | 5.38(dd) | 139.12 CH |
| 13 | 2.20(m) | 37.98 CH | 2.20(m) | 37.99 CH |
| 14 | 0.98(d) | 21.63 CH₃ | 0.98(d) | 21.51 CH₃ |
| 15 | 1.31(m) | 38.19 CH₂ | 1.31(m) | 38.30 CH₂ |
| 16 | 2.00(q) | 26.99 CH₂ | 2.00(q) | 27.01 CH₂ |
| 17 | 5.24(t) | 130.20 CH | 5.23(t) | 129.48 CH |
| 18 | | 130.63 C | | 131.89 C |
| 19 | 1.71(s) | 16.68 CH₃ | 1.71(s) | 16.38 CH₃ |
| 20 | 2.62(d), | 42.53 CH₂ | 2.46(d), | 45.81 CH₂ |
| | 2.20(m) | | 2.20(m) | |
| 21 | 4.76(d) | 79.20 CH | 4.21(dd) | 70.84 CH |
| 22 | | 177.80 C | | 177.92 C |
| 23 | | 96.80 C | | 177.92 C |
| 24 | 1.64(s) | 6.11 CH₃ | | 177.92 C |
| 25 | | 177.60 C | | |

3. Origin of the Preferred Compounds of the Invention

Although the isolation described above of the compounds was conducted from Ircinia dendroides organic solvent extract, we have identified by HPLC/MS method the same pounds in several samples of other Ircinia species as Ircinia variabilis and Ircinia oros.

### EXPERIMENTAL SECTION

Animal Material

The sponge was collected in 2000 at Menorca sea (Spain). The sponge forms irregular strap-like branches, the surface is conculose with fine protruding fibers, and it is flexible and very difficult to tear. The sponge is deep brownish-green in life and cream in preservative, The skeleton consists of large yellow plate-like fibers with embedded detritus, and the sesohyl is permeated with distinctive sponging filaments.

The sample is Ircinia dendroides (Poléjaeff) (order Dictyoceratida, family Irciniidae) first described from the Philippines, A voucher specimen has been deposited of the Natural History Museum, London, U.K. (BMNG 1992.10.1.1).

Extraction and Isolation

A sample of frozen sponge (300 gr wet weight) was cut into small pieces (2 cm) and triturated, the marine material was extracted with isopropanol (1.5L×2). After decantation, the combined extracts were concentrated to give a brown solid (5.1 gr) and partitioned between diethylether and water. The organic layer was concentrated to give a brown oil (1.8 gr). The oil was chromatographed on splica gel with a stepwise gradient solvent system: DCM/MeOH (100:1), DCM/MeOH (75:1), DCM/MeOH (50:1), DCM/MeOH (25:1). Firstly, compound 1 (Palinurin) was isolated after this chromatography purification and the compound 2 (Tricantin) was isolated employing HPLC (RP-18, ACN-H₂O) separation.

Compound 1 has been previously described [G. Alfano, G. Cimino and S. De Stefano. Experientia. 1979, 35, 1136-1137]

Compound 2:

pale brown oil (1.8 mg);

UVλₘₐₓ: 241 nm,

IRvₘₐₓ: 2927, 2856, 1724 cm,

ES+(20 eV) M/z=399 (M+H).

¹H-NMR (CD₃OD, 400 MHz, δ ppm): 7.38 (bts, 1H), 7.25 (brs, 1H), 6.29 (brs, 1H), 6.21 (dd, 1H, J=8 Hz, J=15 Hz), 5.77 (d, 1H, J=BHz), 5.38 (dd, 1H, J=8 Hz, J=15 Hz), 5.23 (t, 1H, J=7 Hz), 4.21 (dd, 1H, J=4.7 Hz, J=8.4 Hz), 2.46 (dd, 1H, J=4.7 Hz, J=13.5 Hz), 2.38 (t, 2H, J=7.6 Hz), 2.30 (dd, 1H, J8.4 Hz, J=13.5 Hz) 2.20 (m, 1H, 2.10 (t, 2H, J=7.6 Hz), 2.00 (q, 2H, J=7 Hz), 1.71 (s, 3H), 1.69 (m, 2H), 1.68 (s, 3H), 1.31 (m, 2H), 0.98 (d, 3H). ¹³C-NMR (CD₃OD, 100 MHz, δ ppm): 178.0, 144.0, 140.1, 139.1, 136.8, 131.9, 129.4, 126.7, 126.6, 126.3, 11.9, 70.92, 45.8, 40.4, 38.3, 37.9, 30.8, 29.5, 27.0, 25.2, 21.5, 16.5, 16.7.

Materials and Methods

HPLC was performed with a symmetry C18 (4.6×150 mm, 3.5 cm) column using a Waters Alliance 2695 with a 2996 photodiode array and ZQ2000 mass spectrometer used for the analytical separation and for UV and mass determination. The gradient used for the elution was 0-5 min 80% A: 20% B; 5-40-100% B; 40-45 min 100% B; 45-46 min 80% A: 20% B (A=H₂O 0.1% HF; B=AcN 0.1% HF). IR Spectra were recorded on a Brulcer Tensor 27 FT-IR spectrometer,

¹H, ¹³C, NMR spectra were recorded on a Vaian AS 400 spectrometer in CD₃OD using the solvent as a reference standard (¹H, 4.87, 3.31, and ¹³C, 49.1). ¹H, ¹³C, COSY, HSQC, HMBC, NOE and DEPT spectra were obtained using standard Varian pulse sequences.

1. The use of a compound of formula I: 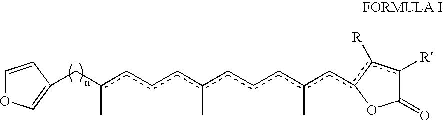
wherein:
n is 0, 1, 2, or 3
the bonds shown with the dashed lines are saturated, or unsaturated with one or more double bonds;
R, and R′ are independently selected from hydrogen, alkyl, aryl, —OH, —OR″, —SH, —SR″, —NH₂, —NHR″, ═O, ═NH, ═NR″;
R″ is independently selected from alkyl, aryl,
in the preparation of a medicament for the treatment of a disease requiring a GSK-3 inhibitor.

2. Use according to claim 1, wherein the compound of formula I is a compound of formula II:

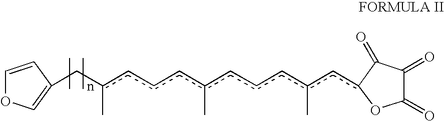

3. Use according to claim 1, wherein the compound of formula I is a compound 1:

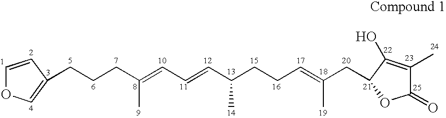

4. Use according to claim 1, wherein the compound of formula I is compound 2:

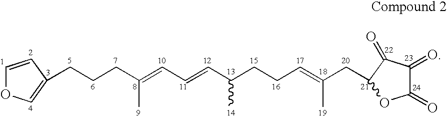

5. The use of claim 1, wherein the disease is Alzheimer's disease, or a tauopathy (Corticobasal degeneration, Pick's disease, supranuclear palsy, etc.), bipolar disorder, diabetes type II, hyperproliferative disease such as cancer, displasais or metaplasais of tissue, psoriasis, arteriosclerosis or restenosis, or chronic inflammatory process.

6. The use according to claim 1, where the diseases is a chronic neurodegenerative condition including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, taupathies such as Pick's disease, corticobasal degeneration, supranuclear palsy, etc., frontotemporal dementia, Huntington's disease, AIDS associated dementia, amyotrophic lateral sclerosis, multiple sclerosis, diabetes, especially diabetes type II, and conditions associated with diabetes, neurotraumatic diseases such as acute stroke, mood disorders such as schizophrenia and bipolar disorders, promotion of functional recovery post stroke, cerebral bleeding (for example, due to solitary cerebral amyloid angioplathy), hair loss, obesity, atherosclerotic cardiovascular disease, hypertension, polycystic ovary syndrome, syndrome X, ischaemia, traumatic brain injury, cancer, leucopenia, Down's syndrome, Lewy body disease, cancer and hyperproliferative diseases as hyperplasias, metaplasias, displasias, and immunodeficiency, psoriasis, arteriosclerosis or restenosis, and inflammation and chronic inflammatory processes.

7. A compound of formula II: 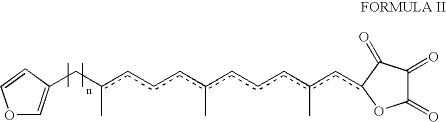
wherein:
n is 0, 1, 2, or 3
the bonds shown with the dashed lines are saturated, or unsaturated with one or more double bonds.

8. A compound according to claim 7, wherein the compound of formula II is compound 2:

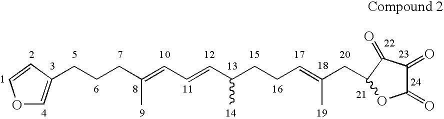

9. A compound of formula II: 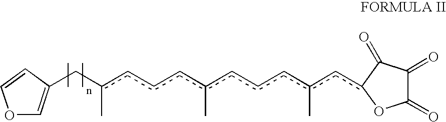
for therapeutic use.

10. A compound of formula 2 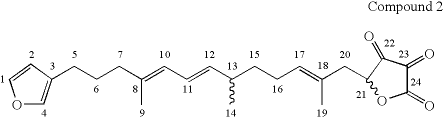
for therapeutic use.

11. A pharmaceutical composition for use as a GSK-3 inhibitor comprising a compound of the formula: 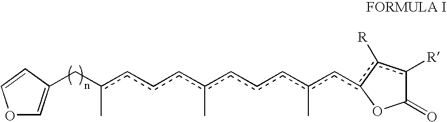
wherein:
n is 0, 1, 2, or 3
the bonds shown with the dashed lines are saturated, or unsaturated with one or more double bonds;
R, and R′ are independently selected from hydrogen, alkyl, aryl, —OH, —OR″, —SH, —SR″, —NH2, —NHR″, ═O, ═NH, ═NR″;
R″ is independently selected from alkyl, aryl,
together with a pharmaceutically acceptable carrier.

12. A pharmaceutical composition according to claim 11, wherein the compound of formula I is a compound of formula II:

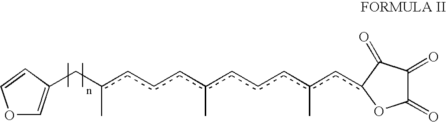

13. A pharmaceutical composition according to claim 11, wherein the compound of formula I is compound 1:

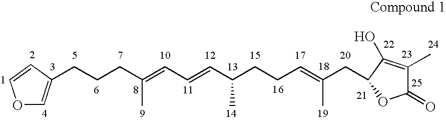

14. A pharmaceutical composition according to claim 11, wherein the compound of formula I is compound 2:

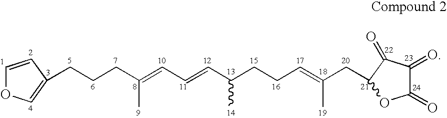

15. A method of treating Alzheimer's disease, or a tauopathy (Corticobasal degeneration, Pick's disease, supranuclear palsy, etc.), bipolar disorder, diabetes type II, hyperproliferative disease such as cancer, displasais or metaplasais of tissue, psoriasis, arteriosclerosis or restenosis, or chronic inflammatory process, which comprises administering a compound of formula I: 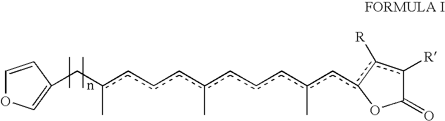
wherein:
n is 0, 1, 2, or 3
the bonds shown with the dashed lines are saturated, or unsaturated with one or more double bonds;
R, and R′ are independently selected from hydrogen, alkyl, aryl, —OH, —OR″, —SH, —SR″, —NH2, —NHR″, ═O, ═NH, ═NR″;
R″ is independently selected from alkyl, aryl.

16. A method for treating chronic neurodegenerative conditions including dementias such as Alzheimer's disease, Parkinson's disease, progressive supranuclear palsy, subacute sclerosing panencephalitic parkinsonism, postencephalitic parkinsonism, pugilistic encephalitis, guam parkinsonism-dementia complex, taupathies such as Pick's disease, corticobasal degeneration, supranuclear palsy, etc., frontotemporal dementia, Huntington's disease, AIDS associated dementia, amyotrophic lateral sclerosis, multiple sclerosis, diabetes, especially diabetes type II, and conditions associated with diabetes, neurotraumatic diseases such as acute stroke, mood disorders such as schizophrenia and bipolar disorders, promotion of functional recovery post stroke, cerebral bleeding (for example, due to solitary cerebral amyloid angioplathy), hair loss, obesity, atherosclerotic cardiovascular disease, hypertension, polycystic ovary syndrome, syndrome X, ischaemia, traumatic brain injury, cancer, leucopenia, Down's syndrome, Lewy body disease, cancer and hyperproliferative diseases as hyperplasias, metaplasias, displasias, and immunodeficiency, psoriasis, arteriosclerosis or restenosis, and inflammation and chronic inflammatory processes, which comprises administering a compound of formula I: 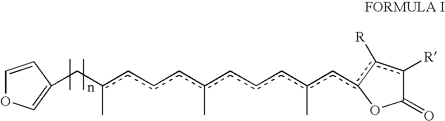
wherein:
n is 0, 1, 2, or 3
the bonds shown with the dashed lines are saturated, or unsaturated with one or more double bonds;
R, and R′ are independently selected from hydrogen, alkyl, aryl, —OH, —OR″, —SH, —SR″, —NH2, —NH₂, ═O, ═NH, ═NR″;
R″ is independently selected from alkyl, aryl.
